(19) United States
(12) Patent Application Publication
(10) Pub. No.: US 2024/0325320 A1
(43) Pub. Date: Oct. 3, 2024

(54) THERAPEUTIC LIQUID COMPOSITIONS

(71) Applicant: JOHNSON & JOHNSON CONSUMER INC., Skillman, NJ (US)

(72) Inventors: Gregory E. Koll, Hillsborough, NJ (US); Jonathan Zeszotarski, Ambler, PA (US); Gersi Hoxha, Fort Washington, PA (US)

(21) Appl. No.: 18/615,156

(22) Filed: Mar. 25, 2024

Related U.S. Application Data
(60) Provisional application No. 63/493,395, filed on Mar. 31, 2023.

(51) Int. Cl. A61K 31/135; A61K 9/00; A61K 31/451; A61K 31/4545; A61K 31/495; A61K 47/10; A61K 47/12

(52) U.S. Cl. CPC A61K 31/135; A61K 9/0095; A61K 31/451; A61K 31/4545; A61K 31/495; A61K 47/10; A61K 47/12

(57) ABSTRACT

Therapeutic liquid compositions useful in the treatment of various conditions comprising a bulk sweetener derived from a grain or fruit source.

### CROSS-REFERENCE TO RELATED APPLICATIONS

This application claims the benefit of U.S. provisional application 63/493,395 filed on Mar. 31, 2023, the complete disclosure of which is hereby incorporated herein by reference for all purposes.

### FIELD OF THE INVENTION

The present invention relates to liquid compositions, mores specifically to therapeutic liquid compositions. The present invention also relates to a process for manufacturing the liquid compositions and to methods for alleviating symptoms in human subjects upon administration of the liquid compositions. The liquid compositions, which can comprise one or more therapeutically active agents, are particularly useful in treatment of allergy symptoms, pain symptoms, and cough and cold symptoms, including but not limited to pain, cough, nasal congestion, rhinitis and sore throat.

### BACKGROUND OF THE INVENTION

Orally administered medicaments or pharmaceuticals are given to patients in many forms, including solid forms, such as, capsules, caplets, gel caps, or tablets, and liquid forms, such as, solutions, e.g., syrups and elixirs, emulsions, or suspensions. Children, older patients, and many other patients, including disabled or incapacitated patients, have trouble swallowing solid forms, e.g., whole tablets and even capsules. Therefore, in cases where the dosage to be administered cannot be made into a very small tablet or capsule, it is desirable to provide the medicine either in a chewable solid form or a liquid form. For many patients, including pediatric and geriatric patients, a liquid oral dosage form is preferable over chewable dosage form because of the ready swallowability without chewing of the liquid dosage form.

Suspensions are a two-phase system having solid substantially water insoluble active agent particles dispersed throughout liquid medium. A suspension does not encompass emulsions, which are meant to describe liquids suspended within liquid carriers or syrup formulations containing only substantially fully dissolved pharmaceutical active agents. As used herein, a “particle” may be a crystal, a granule, an agglomerate, or any undissolved solid material. The particles of the present invention preferably have a median particle size (d50%) of from about 2 to about 150 microns, more preferably from about 10 to about 100 microns. The challenges of keeping the substantially water insoluble active agent suspended, assuring stability of the substantially water insoluble active agent, and maintaining dose uniformity for a prolonged period of time, have been previously addressed. See for example, U.S. Pat. Nos. 5,409,907, and 5,374,659.

In a pharmaceutical suspension, typically at least one active agent is present substantially in the form of undissolved solid particles, i.e., the substantially water insoluble active agent. However, in any such system, a portion of such active agent may be in the dissolved state. In formulating such systems, it is advantageous to minimize the amount of drug present in the dissolved state. Minimizing the amount of active agent in solution is advantageous for both the taste and the chemical and/or physical stability of the product.

A liquid dosage form may also be present in the form of a solution, where the therapeutic ingredient or ingredients are substantially dissolved in the liquid blend. When the dose of the therapeutic ingredient is less than 50 g/5 mL, the therapeutic ingredient may be dissolved in the form of a solution.

A common problem associated with liquid dosage forms is the often disagreeable taste of the active agents that manifest during the time that the liquid dosage form is in the mouth prior to swallowing. While suspensions typically offer superior taste masking to other liquid forms, those skilled in the art are aware of the considerable technical difficulties in producing a stable and organoleptically acceptable suspension.

Further, the solutions to such challenges must be re-evaluated when over-the-counter (“OTC”) oral solution or oral suspension products are formulated or re-formulated to use reduced ingredients or naturally sourced ingredients. The term “clean” or “clean medicine” is a newly emerging trend for OTC pharmaceutical products. Although there is no generally accepted definition for the term “clean”, it is generally understood by consumers to mean any of the following: no artificial flavors, dyes, preservatives or sweeteners; sourced from nature; and/or no artificial, chemical or unnecessary ingredients.

Formulating “clean” oral solution or oral suspension products may limit or exclude certain ingredients that have been traditionally used in pharmaceutical suspensions to overcome the challenges of keeping the substantially water insoluble active agents suspended, assuring stability of the substantially water insoluble active agents, and maintaining dose uniformity for a prolonged period of time. New ingredients that fit the definition of “clean” must be identified and evaluated to ensure that such newly formulated pharmaceutical solution or suspension products meet the safety and efficacy standards that are required of all OTC pharmaceutical products.

Dyes have been traditionally added to pharmaceutical suspensions for elegance and to mask discoloration. However, it has been found that some patients develop or have allergies to or are sensitive to dyed suspensions. Dyed suspensions may further stain clothing, furniture, carpeting, and the like when spilled. Therefore, dye-free suspensions are very desirable.

Coloring agents are often added to pharmaceutical liquid products to produce pharmaceutically acceptable characteristics, to provide an identifying factor and also to provide consistency among the batches of a product. Often the color of the excipients that are used to manufacture an OTC product may contribute to products that look inconsistent or “off-color” to a patient. The patient or consumer may perceive the inconsistency or color change as an indication that the product was improperly manufactured, has expired, or is generally ineffective, resulting in rejection of the product.

In many cases liquid dosage forms include solvents or carriers to dissolve the active ingredient. Common liquid solvents include propylene glycol or polyethylene glycol. Some of these solvents and carriers are artificial in nature or undergo significant chemical processing, even if they are derived from natural sources. Common bulk sweeteners include sugar alcohols in solution such as sorbitol solution. In many cases these solvents and bulk sweeteners are from an artificial origin and not from natural plant sources, which many patients now prefer.

Even where solvents and bulk sweeteners are derived from natural plant sources, these ingredients often have high sugar content and/or a high glycemic index, which may be undesirable to consumers; in particular, young children, diabetic or pre-diabetic patients, or patients who are generally health conscious. For example, US20210290532A1 from GE Healthcare discloses a stable pharmaceutical formulation or suspension that has a pharmaceutical active agent, agave, and a dilutant. This formulation or suspension has viscosity suitable for drinking, however, may have a high glycemic index due to the incorporation of agave syrup, which has a high fructose content.

Additionally, naturally derived solvents and bulk sweeteners may be difficult to incorporate, stabilize or preserve in an oral suspension formulation. An example of a natural bulk sweetener is disclosed in US20080311272 (A1) from Wild Valencia S.A. which describes a composition comprising carbohydrates obtainable from carob and carbohydrates obtainable from at least one further fruit, including apple and grape. This natural bulk sweetener or similar bulk sweetener is sold by ADM Company under the tradename of “Fruit-Up®.”

Thus, there remains a need for liquid medication compositions that comprise inactive materials which are derived from natural sources with relatively low glycemic indices. Such a composition should also work quickly and provide superior relief for an extended period of time.

### SUMMARY OF THE INVENTION

The present invention is directed to liquid compositions that can be used to alleviate conditions such as those associated with allergy, cough cold, digestive health or pain by carrying an active therapeutic ingredient. This active therapeutic ingredient may be a pharmaceutical active ingredient, homeopathic ingredient, herbal ingredient or nutritional supplement. These compositions would comprise a substantial amount of inactive ingredients that are of natural origin or natural sources.

According to one example, a liquid composition may include at least one therapeutic ingredient and at least one natural bulk sweetener, where the fructose content of the total amount of natural bulk sweetener in the composition is less than 75 percent.

According to an example, a liquid composition may include a therapeutic ingredient and a natural bulk sweetener that has a fructose content of less than 50 percent of the natural bulk sweetener. The natural bulk sweetener may be selected from glycerin, allulose syrup, rice syrup, tapioca syrup, apple syrup, carob syrup, and combinations thereof. The liquid composition may have a total fructose level of less than 0.7 g/mL.

According to one example, the therapeutic ingredient may be a pharmaceutical active ingredient. According to other examples, the therapeutic ingredient may be a homeopathic active ingredient and/or a nutritional supplement. In a further example, the therapeutic ingredient may be a pharmaceutical active ingredient combined with a homeopathic active ingredient and/or a nutritional supplement.

The liquid composition may be in the form of a suspension or a solution.

In one example, the pharmaceutical active ingredient may be selected from the group consisting of acetaminophen, diphenhydramine, dextromethorphan, guaifenesin, ibuprofen, cetirizine, loperamide and phenylephrine. For example, the therapeutic ingredient may be an antihistamine.

In a further example, the liquid composition may further include a sensate.

The liquid composition may include a second natural bulk sweetener. In one such example, the natural bulk sweetener may be glycerin and the second natural bulk sweetener may be tapioca syrup. In another example, the natural bulk sweetener may be glycerin and the second natural bulk sweetener may be brown rice syrup. In another example, the natural bulk sweetener may be glycerin and the second natural bulk sweetener may be allulose syrup.

In another example the natural bulk sweetener may be glycerin and the second natural bulk sweetener may be a combination of apple syrup and carob syrup. In one example, the ratio of the combination of apple syrup and carob syrup to glycerin is about 3.30:1.

In one example, the liquid composition may be substantially free or completely free of agave syrup. In another example, the liquid composition may be substantially free of any ingredient having a fructose content greater than 15 percent.

According to another aspect of the invention, there may be a method of alleviating a symptom selected from the group consisting of pain, fever, cough, nasal congestion, diarrhea, allergic rhinitis and sore throat in a subject by administering compositions as disclosed herein.

### DETAILED DESCRIPTION OF INVENTION

It is believed that one skilled in the art can, based upon the description herein, utilize the present invention to its fullest extent. The following specific examples and embodiments are to be construed as merely illustrative, and not as limiting the remainder of the disclosure in any way whatsoever.

Unless defined otherwise, all technical and scientific terms used herein have the same meaning as commonly understood by one of ordinary skill in the art to which the invention belongs.

Unless otherwise indicated, all documents cited are, in relevant part, incorporated herein by reference; the citation of any document is not to be construed as an admission that it is prior art with response to the present invention. Furthermore, all documents incorporated herein by reference are only incorporated herein to the extent that they are not inconsistent with this specification.

As used herein, all percentages are by weight unless otherwise specified. In addition, all ranges set forth herein are meant to include any combinations of values between the two endpoints, inclusively.

As used herein, the term “active ingredient” is used herein in a broad sense and may encompass any material that imparts a therapeutic effect. For example, the active ingredient can be a pharmaceutical, nutraceutical, vitamin, dietary supplement, nutrient, herb, foodstuff, dyestuff, nutritional, mineral, supplement, oral care agent or flavoring agent (sensate) or the like and combinations thereof.

“Dosage form” applies to any composition designed to contain a specific pre-determined amount (dose) of a certain ingredient, and for example, an active ingredient as defined herein. Suitable dosage forms may be pharmaceutical drug delivery systems and systems for delivering minerals, vitamins and other nutraceuticals, oral care agents, flavorants, sensates, and the like. In a particularly preferred example, the dosage form is an orally administered system for delivering a pharmaceutical active ingredient to the gastro-intestinal tract of a human.

“Liquid dosage forms” may nonexclusively include suspensions, syrups, solutions or elixirs, wherein one or more of the active ingredients is dissolved, partially dissolved or in an undissolved or suspended state.

“Therapeutic effect,” means any effect or action of an active ingredient intended to diagnose, treat, cure, mitigate, or prevent disease, or affect the structure or any function of the body.

Unless otherwise indicated, percentages used to express amounts of ingredients are percentage by weight (referred to as “weight %,” “wt %”, “% by weight” or “% (w/volume)”). Similarly, weight ratios used to express relative proportions of ingredients are also determined using percentage by weight (i.e., weight ratios are calculated by dividing the percentage by weight of one ingredient by another). Unless stated otherwise, all ranges are inclusive of the endpoints, e.g., “from 4 to 9” includes the endpoints 4 and 9.

As used herein, the term “safe and effective amount” means an amount sufficient to induce the desired effect, but low enough to avoid serious side effects. The safe and effective amount of the compound, extract, or composition will vary with, e.g., the age, health and environmental exposure of the end user, the duration and nature of the treatment, the specific extract, ingredient, or composition employed, the particular carrier utilized, and like factors.

As used herein, the term “about” refers to within 5% weight, within 4% weight, within 3% weight, within 2.5% weight, within 2% weight, or within 1% weight of a disclosed value.

In certain embodiments, the present invention as disclosed herein may be practiced in the absence of any compound, method step, or element (or group of compounds or elements) which is not specifically disclosed herein.

### Active Ingredient(s)

The dosage form of the present invention preferably contains one or more (or at least one) active ingredients. Suitable pharmaceutical active ingredients include analgesics, anti-inflammatory agents, antiarthritics, anesthetics, antihistamines, antitussives, antibiotics, anti-infective agents, antipyretics, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, cough suppressants, decongestants, expectorants, oral contraceptives, diuretics, gastrointestinal agents, migraine preparations, motion sickness products, mucolytics, muscle relaxants, osteoporosis preparations, polydimethylsiloxanes, respiratory agents, sleep-aids, urinary tract agents, and pharmaceutically acceptable salts thereof, derivatives thereof, combinations thereof and mixtures thereof.

In accordance with one example, the active ingredient may be selected from (or selected from the group consisting of) acetyl salicylic acid, acetic acid derivatives such as indomethacin, diclofenac, sulindac, and tolmetin; fenamic acid derivatives such as mefanamic acid, meclofenamic acid, and flufenamic acid; biphenylcarbodylic acid derivatives such as diflunisal and flufenisal; and oxicams such as piroxicam, sudoxicam, isoxicam, and meloxicam; and pharmaceutically acceptable salts thereof, derivatives thereof, combinations thereof and mixtures thereof.

Examples of useful NSAIDs include (as may be selected from or selected from the group consisting of) ibuprofen, naproxen, benoxaprofen, naproxen sodium, fenbufen, flurbiprofen, fenoprofen, fenbuprofen, ibuprofen, ketoprofen, indoprofen, pirprofen, carpofen, oxaprofen, pranoprofen, microprofen, tioxaprofen, suprofen, alminoprofen, tiaprofenic acid, fluprofen, bucloxic acid, celecoxib, and pharmaceutically acceptable salts thereof, derivatives thereof, combinations thereof and mixtures thereof.

The composition of the present invention may also comprise acetaminophen. As used herein, the term “APAP” means acetaminophen or N-acetyl para-aminophenol, including, but not limited to, pharmaceutically acceptable salts, esters, or derivatives thereof.

Examples of cough and cold pharmaceutical active ingredients, which include antihistamines, cough suppressants, decongestants and expectorants, include (or may be selected from or selected from the group consisting of), but are not limited to, bromopheniramine, carbinoxamine, acetylcysteine, guaifenesin, carbocysteine, chlorcyclizine, dexbrompheniramine, bromhexane, phenindamine, pheniramine, pyrilamine, thonzylamine, pripolidine, ephedrine, phenylephrine, pseudoephedrine, phenylpropanolamine, chlorpheniramine, dextromethorphan, diphenhydramine, doxylamine, astemizole, terfenadine, fexofenadine, naphazoline, oxymetazoline, montelukast, propylhexadrine, triprolidine, clemastine, acrivastine, promethazine, oxomemazine, mequitazine, buclizine, bromhexine, ketotifen, terfenadine, ebastine, oxatamide, xylomeazoline, loratadine, desloratadine, noscapine, clophedianol, menthol, benzonatate, ethylmorphone, codeine, acetylcysteine, carbocisteine, ambroxol, belladona alkaloids, sobrenol, guaiacol and cetirizine; and pharmaceutically acceptable salts thereof, derivatives thereof, combinations thereof and mixtures thereof.

Preferably, the at least one active ingredient is an NSAID and/or acetaminophen, and pharmaceutically acceptable salts thereof.

Examples of suitable gastrointestinal agents include antacids such as (or as may be selected from or selected from the group consisting of) calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotidine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate, loperamide and racecadotril; glycopyrrolate; antiemetics, such as ondansetron, analgesics, such as mesalamine.

Examples of suitable polydimethylsiloxanes, which include (as may be selected from or selected from the group consisting of), but are not limited to dimethicone and simethicone, as disclosed in U.S. Pat. Nos. 4,906,478, 5,275,822, and 6,103,260, the contents of each which is expressly incorporated herein by reference. As used herein, the term “simethicone” refers to the broader class of polydimethylsiloxanes, including but not limited to simethicone and dimethicone.

The therapeutically effective amount of active ingredient or active ingredients can readily be determined by one skilled in the art.

Suitable sensates include (or may be selected from or selected from the group consisting of) menthol, peppermint, mint flavors, fruit flavors, chocolate, vanilla, bubblegum flavors, coffee flavors, natural flavors, liqueur flavors and combinations and the like.

In accordance with an example, the composition comprises a polymer also known as a muco-adhesive material. Suitable polymers include gelling material, such as hydrocolloid(s), e.g., (or may be selected from or selected from the group consisting of) pectins, cellulosics, alginates, carrageenans, etc.

In an example, the muco-adhesive material is a thickener. Suitable thickeners include (or may be selected from or selected from the group consisting of), but are not limited to carboxymethylcellulose (CMC), microcrystalline cellulose and blends of CMC and microcrystalline cellulose or mixtures thereof. Some suitable thickeners are of natural origin, including xanthan gum and/or konjac gum. Other thickeners include cellulosic polymers such as hypromellose, hydroxypropylcellulose, hdyroxyethylcellulose and mixtures thereof.

In accordance with an example, the muco-adhesive material includes, but is not limited to, Avicel BV 2219, Avicel CL 611, Avicel Plus® LM 310 stabilizer, 40% solids, Avicel RC 591, Carbopol 971P, Aqualon® CMC-7M8SF-PH, HPMC E4M and Nutricol BV 5616.

The composition may contain from about 0.05% weight to about 3% weight polymer. The composition may contain from about 0.2% weight to about 2% weight polymer. These materials, which improve muco-adhesion, increase duration of relief.

In yet another example, the composition may contain a surface active agent (also known as a surfactant). Suitable surfactants may include polyoxyethylene-polyoxypropylene block copolymers. In an example, the surfactant includes, but is not limited to, Lutrol F68 (Poloxamer® 188); Lutrol F127 (Poloxamer® 407), Tween 80 and mixtures thereof.

The composition may contain from about 0.05 wt. % to about 2 wt. % of surfactant. The composition may contain from about 0.1 wt. % to about 1.5 wt. % of surfactant. The composition may contain from about 0.5 wt. % to about 1 wt. % of surfactant. The surfactant further enhances the perceived coating effect of the composition.

The resulting composition may be delivered as a spray or a liquid. In an example, the composition may include an active ingredient. In another example, the composition may include a sensate.

### Bulk Sweeteners

For the composition of the present invention, a substantial portion of the bulk liquid comprises a natural bulk sweetener. As used herein a “natural bulk sweetener” is defined as one that is derived from a plant source. The natural bulk sweetener may also have additional properties such as one that is from vegan sources, organic sources, non-GMO sources, or is substantially free or completely free of gluten or alcohol. As used herein substantially free is defined as less than 0.1 percent, preferably less than 0.05 percent, preferably less than 0.001 percent, weight by volume. The natural bulk sweetener may be used in the present composition at from about 20 wt. % to about 99 wt. % by volume of the total composition.

The bulk sweetener, or natural bulk sweetener, of the present invention may also have a low glycemic load, as well as a specific level of fructose. For example, the bulk sweetener may have a fructose content less than 75% weight, less than 70% weight, less than 50% weight, less than 40% weight, less than 30% weight, less than 25% weight, less than 20% weight, or less than 15% weight. In another example, the total fructose content of the liquid composition may be less than 0.9 g/mL, or less than 0.7 g/mL. In another example, the composition may be substantially free or completely free of fructose. As used herein substantially free is defined as less than 0.1 percent, preferably less than 0.05 percent, preferably less than 0.001 percent, weight by volume. In another example, the total amount of sugar in the composition may be less than 0.9 g/mL or less than 0.7 g/mL.

Suitable natural bulk sweeteners for the present invention include (or may be selected from or selected from the group consisting of) but are not limited to allulose syrup, rice syrup, tapioca syrup; and apple and carob syrups and mixtures thereof, such as the combination of apple syrup and carob syrup sold commercially by the ADM Company under the tradename of “Fruit-Up®”.

Other natural bulk sweeteners may include glycerin. Glycerin may be present in the composition from about 10% weight to about 98% weight of the composition. Glycerin may also be combined with a different (or second) natural bulk sweetener.

The composition may comprise a mixture of one or more of the natural bulk sweeteners in order to balance the sweetness and flavor profile of the final composition. An amount of first natural bulk sweetener may be added at from about 10% weight to about 90% weight of the composition, or from about 20% weight to about 50% weight of the composition; and an amount of second natural bulk sweetener may be added from about 10% weight to about 90% weight of the composition, or from about 20% weight to about 50% weight of the composition. The composition may also comprise a third natural bulk sweetener (i.e., a natural bulk sweetener different from the first and second natural bulk sweeteners), where an amount of third bulk sweetener is added from about 5% weight to about 30% weight of the composition.

According to one example, the natural bulk sweetener may be a combination of glycerin and tapioca syrup. The glycerin may be present in the composition in a range of about 20 wt. % to about 40 wt. %, or about 25 wt. % to about 35 wt. %, for example, about 30 wt. %. The tapioca syrup may be present in the composition in a range of about 50 wt. % to about 80 wt. %, or about 60 wt. % to about 75 wt. %, for example, about 67 wt. %, or about 69 wt. %, or about 70 wt. %. A ratio of the tapioca to glycerin may be from about 2:1 to about 3:1, or about 2.1:1 to about 2.5:1, or about 2.3:1.

According to an example, the natural bulk sweetener may be a combination of glycerin and brown rice syrup. The glycerin may be present in the composition in a range of about 20 wt. % to about 40 wt. %, or about 25 wt. % to about 35 wt. %, for example, about 30 wt. %. The brown rice syrup may be present in the composition in a range of about 40 wt. % to about 60 wt. %, or about 45 wt. % to about 55 wt. %, for example, about 50 wt. %, or about 49 wt. %. A ratio of the brown rice syrup to glycerin may be from about 1:1 to about 2:1, or about 1.4:1 to about 1.8:1, or about 1.6:1.

According to another example, the natural bulk sweetener may be a combination of glycerin and allulose syrup. The glycerin may be present in the composition in a range of about 20 wt. % to about 40 wt. %, or about 25 wt. % to about 35 wt. %, for example, about 30 wt. %. The allulose syrup may be present in the composition in a range of about 50 wt. % to about 90 wt. %, or about 60 wt. % to about 80 wt. %, or about 65 wt. % to about 75 wt. %, for example, about 70 wt. %, or about 69 wt. %. A ratio of the allulose syrup to glycerin may be from about 2:1 to about 3:1, or about 2.1:1 to about 2.5:1, or about 2.3:1.

According to another example, the natural bulk sweetener may be a combination of glycerin, apple syrup and carob syrup. The combination of apple syrup and carob syrup may be identical or similar to the product commercially sold under the trademark Fruit-Up® by Archer Daniels Midland (“ADM”) Company, and/or as described in European Patent EP2002734B1 titled “Carbohydrate Composition Obtainable from Mediterranean Fruits,” which is incorporated herein by reference, namely, the apple and carob syrups may be incorporated such that 35-60%, optionally 35-50%, optionally 35-45%, optionally 35-40%, optionally 35% (w/w) carbohydrates are obtainable from carob, 35-50%, optionally 35-45%, optionally 35-40%, optionally 40% (w/w) carbohydrates are obtainable from at least one further fruit (e.g., apple), wherein the percentages are based on the dry matter.

In one example, the glycerin may be present in the composition in a range of about 20 wt. % to about 40 wt. %, or about 25 wt. % to about 35 wt. %, for example, about 30 wt. %. The combination of apple syrup and carob syrup, for example as commercially sold under trademark Fruit-Up® by ADM Company, may be present in the composition in a range of about 50 wt. % to about 90 wt. %, or about 60 wt. % to about 80 wt. %, for example, about 68 wt. %, about 69, or about 70 wt. %. A ratio of the combination of apple syrup and carob syrup, for example as commercially sold under trademark Fruit-Up® by ADM Company, to glycerin may be from about 2:1 to about 3:1, or about 2.1:1 to about 2.5:1, for example, about 2.3:1. In another example, the ratio of the combination of apple syrup and carob syrup, for example as commercially sold under trademark Fruit-Up® by ADM Company, to glycerin may be from about 2:1 to about 4:1, or about 3:1 to about 4:1, or about 3:1 to about 3.5:1, for example, about 3.30:1.

The composition may comprise a natural bulk sweetener plus a sugar alcohol solution. Suitable sugar alcohol solutions include (or may be selected from or selected from the group consisting of), but are not limited to, sorbitol, maltitol, mannitol, xylitol and erythritol. An amount of first natural bulk sweetener may be added at from about 10% weight to about 90% weight of the composition, or from about 20% weight to about 50% weight of the composition; and an amount of second sugar alcohol solution may be added from about 10% weight to about 90% weight of the composition, or from about 20% weight to about 50% weight of the composition. Alternatively, the composition may be substantially free of a sugar alcohol solution.

In one example, the composition may include glycerin and an additional bulk sweetener derived from a grain or fruit source. In this example, glycerin may be present from 10 wt. % to 40 wt. % of the composition and the additional bulk sweetened may be present from 10 wt. % to 40 wt. % of the composition. Glycerin may be added to mitigate the unpleasant taste or numbing of the pharmaceutical active ingredient.

In one example, the weight ratio of glycerin to an additional plant-based syrup is from 1:3 to about 3:1, or about 1:2 to about 2:1. In this example, the additional plant-based syrup is selected from (or selected from the group consisting of) rice, brown rice, tapioca, carob allulose syrup and mixtures thereof.

In one example, the composition includes five or less ingredients. In another example, the composition includes four or less ingredients. In one version of this example, the composition is limited to diphenhydramine, glycerin, an additional plant-based syrup, and flavor. In this example, the additional plant-based syrup is selected from (or selected from the group consisting of) allulose, carob, rice, brown rice, tapioca syrup and mixtures thereof.

Table 1 shows the sugar content for various bulk sweeteners.

TABLE 1
Sugar content for bulk sweeteners

| | Fructose | Total Carbohydrate |
|---|---|---|
| Sweetener | Content (%) | Content (g/100 g) |
| Allulose Syrup | <0.15% | 71.0 grams |
| Brown Rice Syrup | 0.0 | 79.0 grams |
| Fruit-Up ® (Apple and Carob) Syrup | 10.54%¹ | 57.96 grams |
| Tapioca Syrup | 0.0% | 73.9 grams |
| High Fructose Corn Syrup | 55.0% | 76.0 grams |
| Agave Syrup | 80.0% | 76.0 grams |

¹Reported as 70% dry matter or Brix at 20º C., or the measure of dissolved solids in a liquid

### Preservatives

The composition of the present invention may include a natural preservative, artificial preservative or combinations thereof. Suitable natural preservatives include potassium sorbate and citrus extracts. Suitable artificial preservatives include (or may be selected from or selected from the group consisting of) sodium benzoate, salts of edetate (also known as salts of ethylenediaminetetraacetic acid, or EDTA, such as, disodium edetate), parabens (such as, methyl, ethyl, propyl and butyl p-hydroxybenzoic acids esters) and mixtures thereof. The total preservative may be present from 0.01% weight to 5% weight. For example, the preservative of the composition may be potassium sorbate in an amount of from about 0.1 wt. % to about 0.5 wt. %, or about 0.2 wt. %. The present composition may also be substantially free or completely free of a preservative. As used herein substantially free is defined as less than 0.1 percent, preferably less than 0.05 percent, preferably less than 0.001 percent, weight by volume.

If the composition is substantially free of preservative, the composition also has a water activity of less than 0.7 when tested using a water activity meter. Water activity (aw) of a composition is defined as the ratio between the vapor pressure of the composition, when in a completely undisturbed balance with the surrounding air media, and the vapor pressure of distilled water under identical conditions. Water activity is described in detail later in this description.

In certain examples, the composition may be substantially free or completely free of added water. Added water may be defined as water that is not present in the bulk sweetener or plant based syrup. In some examples, the added water content is less than 5% weight, or less than 2% weight, or less than 1% weight or less than 0.5% weight of added water. In some examples, the total water content in the composition is less than 5% weight, or less than 2% weight, or less than 1% weight or less than 0.5% weight of total water in the composition.

pH Adjusting Agent

The composition may include a pH adjusting agent or a buffering agent, including salts, acids or bases. In certain examples the pH adjusting agent includes anhydrous citric acid or sodium citrate. The pH adjusting agent may be added at a quantity of less than 2% weight, or less than 1% weight or less than 0.5% weight or less than 0.2% by weight, or at about 0.1% by weight.

The pH of the composition may be less than 6 (or about 6), optionally between 5 (or about 5) and 6 (or about 6), for example between 5.8 and 5.9, or about 5.888.

### Thickeners

The suspensions of the present invention may employ suspending systems as known in the art that include, but are not limited to, at least one thickening component. The thickening component may include one or more thickening agents that may be selected from hydrophilic, i.e., water soluble, polymers such as hydrocolloids, swelling or gelling polymers, and the like. In one example, the thickening component may combine the attributes of a structuring agent and a swelling agent. In another preferred example, the thickening component may combine the attributes of at least two structuring agents, e.g., a primary structuring agent and a secondary structuring agent.

A structuring agent, when introduced into an appropriate aqueous environment, may form an ordered structure, believed to be stabilized by hydrogen bonding and molecular entanglement. Hydrocolloids are a particularly good type of structuring agent. Hydrocolloids are dispersions of particles around which water molecules and solvated ions form a shell-like structure, fluid absorption occurs principally by swelling and enlargement of the structure.

Examples of suitable hydrocolloids include (or may be selected from or selected from the group consisting of), but are not limited to, alginates, agar, guar gum, locust bean, carrageenan, tara, gum arabic, tragacanth, pectin, xanthan, gellan, maltodextrin, galactomannan, pusstulan, laminarin, scleroglucan, gum arabic, inulin, karaya, whelan, rhamsan, zooglan, methylan, chitin, cyclodextrin, chitosan, cellulosic polymers such as microcrystalline cellulose, carboxymethylcellulose, and derivatives and combinations thereof. In certain examples of the present invention, useful structuring agents may be selected from (or selected from the group consisting of) the hydrocolloids xanthan gum, microcrystalline cellulose, carboxymethylcellulose, and derivatives, co-precipitates, and combinations thereof. In one particularly useful example, the thickening component may include xanthan gum as a primary structuring agent and a co-processed combination of microcrystalline cellulose and carboxymethylcellulose (such as that commercially available from FMC as Avicel-RC 591) as a secondary structuring agent.

Xanthan gum is a high molecular weight natural carbohydrate, specifically, a polysaccharide. A xanthan gum suitable for use in the present invention is a high molecular weight polysaccharide produced by Xanthomonas campestris. Techniques and strains for producing this polysaccharide are described in U.S. Pat. Nos. 4,752,580 and 3,485,719 (the disclosures of each of which are hereby incorporated by reference). The xanthan gum used in the present invention should have a viscosity in a one percent salt solution of from about 1000 to about 1700 cP (mPa-sec). The one percent solution's viscosity should be measured at 25° C. with an LV model Brookfield Synchro-Lectric viscometer at 60 rpm, no. 3 spindle. Xanthan gum is available from several commercial suppliers such a RT Vanderbilt Company and CP Kelco. Examples of suitable xanthan gums are Keltrol, Keltrol F, Keltrol T, Keltrol TF, Xantural 180 and Vanzan NF-ST.

In one example, the secondary structuring agent used in the present invention may be a dried coprecipitated microcrystal of cellulose and sodium carboxymethylcellulose. Sodium carboxymethyl-cellulose is commonly used as a coprecipitate in microcrystalline cellulose. It is particularly useful if the sodium carboxymethylcellulose is included in the range of from about 8 weight percent to about 19 weight percent of the total weight of the coprecipitated microcrystal of cellulose and sodium carboxymethylcellulose. Useful are microcrystalline cellulose products having in the range from about 8 to about 14 weight percent sodium carboxymethylcellulose. These mixtures as described above are commercially available from a variety of sources, including FMC under the trademark Avicel® CL-611, Avicel® RC-581 and Avicel® RC-591.

The thickening component may be a swelling agent that, when exposed to an appropriate aqueous environment, expands and may interact with the structuring agent. Pregelatinized starch is a particularly good swelling agent. Pregelatinized starch, also known as “instantized” starch, is precooked so that it swells and begins to thicken instantly when added to cold water. One particularly suitable pregelatinized starch is prepared from modified, stabilized and waxy, maize food starch, and commercially available from National Starch Company as Instant Starch, Ultrasperse M.

In certain examples, an optional auxiliary suspending agent may be used. The auxiliary suspending agent may be selected from the group consisting of hydroxyethylcellulose and a pharmaceutically acceptable salt of carboxymethylcellulose. Suitable pharmaceutically acceptable salts of carboxymethylcellulose may include (or may be selected from or selected from the group consisting of) sodium and/or calcium salts of a polycarboxymethyl ether of cellulose, commercially available as sodium carboxymethylcellulose, USP and calcium carboxymethylcellulose, NF. Sodium carboxymethylcellulose, USP contains between about 6.5 to about 7.5% by weight sodium on a dry basis and is commercially available from Aqualon Co. under the product designation Aqualon. The hydroxyethylcellulose is a partially substituted poly (hydroxyethyl) ether of cellulose. Hydroxycellulose, NF is commercially available from Aqualon Co. under the product designation Natrosol.

In examples where the composition is a suspension, the composition may also include at least one thickening agent. In examples where the composition is an oral solution, the composition may be substantially free or completely free of a thickener. As used herein substantially free is defined as less than 0.1 percent, preferably less than 0.05 percent, preferably less than 0.001 percent, weight by volume.

### Additional Sweeteners

The second sweetener may be an artificial sweetener or natural sweetener. Artificial sweeteners include but are not limited to aspartame, neotame, saccharin and sucralose. Natural sweeteners include but are not limited to stevia or steviol glycosides, monk fruit extract, licorice root extract and other derivatives of glycyrrhiza. The additional sweetener may be added from about 0.01 wt. % to about 5 wt. %, or from about 0.1 wt. % to about 3 wt. % of the total composition.

In an example, the composition may further include, as such additional sweeteners, stevia and/or monoammonium glycyrrhizinate. Stevia may be present in the composition in a range of about 0.01 wt. % to about 2 wt. %, or about 0.1 wt. % to about 1 wt. %, for example, about 0.2 wt. %, or about 0.15 wt. %. Stevia may alternatively be present in the composition in higher amounts in a range of about 10 wt. % to about 30 wt. %, for example about 20 wt. %. The monoammonium glycyrrhizinate may be present in the composition in a range of about 0.01 wt. % to about 2 wt. %, or about 0.01 wt. % to about 0.1 wt. %, for example, about 0.04 wt. %.

According to an example, the composition may also exclude certain ingredients. The term “completely free of” refers to a composition having the complete absence of an ingredient.

In an example the composition is free of a bulk sweetener which has a fructose amount of greater than 75 percent, or greater than 70 percent or greater than 50 percent. In another example the total fructose content of the composition is less than 0.9 g/mL, or less than 0.7 g/mL. In another example the total amount of sugar in the composition is less than 0.9 g/mL or less than 0.7 g/mL. The present composition may be substantially free or completely free of agave syrup. The present composition may be substantially free or completely free of corn syrup or high fructose corn syrup. The present composition may be substantially free or completely free of fructose.

The amount of total sugar administered can be determined within a dosing regimen or method of using the composition of the present invention. The amount of sugar in a 24 hour dosing regimen may be less than 60 grams of sugar, or less than 50 grams of sugar, or less than 40 grams of sugar when calculated for dosing the therapeutic amount of the composition. Total sugar is defined as the combined amount of sugars including sucrose, maltose, lactose, glucose and fructose.

In one embodiment the composition is substantially free of aspartame, neotame, saccharin and sucralose. In another embodiment the composition is substantially free of stevia or steviol glycosides, monk fruit extract, licorice root extract and other derivatives of glycyrrhiza.

### Density

The density of the composition may be from about 1.00 g/mL to about 1.50 g/mL, or from about 1.20 g/mL to about 1.40 g/mL or from about 1.25 to about 1.35 g/mL.

### Stability

The invention of the present composition may be stable when stored at accelerated conditions or ambient conditions for up to 24 months, or, under such conditions, up to 1 month, or up to 3 months, or up to 6 months, or up to 12 months, or up to 18 months. Accelerated conditions may include storage at 30° C., 40° C., 50° C., 60° C. and under freeze-thaw conditions. Stability includes physical stability such as but not limited to viscosity, water activity and density; chemical stability such as the degradation of the active ingredient, and microbial stability using antimicrobial effectiveness testing (AET).

### Water Activity

Water activity (aw) is a measurement of the energy status of the water in a system. It indicates how tightly water is “bound”, structurally or chemically, within a substance. Water activity is the relative humidity of air in equilibrium with a sample in a sealed measurement chamber. Water activity is defined as the ratio of vapor pressure of water in the sample to vapor pressure of pure water at the same temperature. The concept of water activity is of particular importance in determining product quality and safety.

Water activity is the best index for microbial growth. A product may contain a relatively large percentage of moisture, but if the water is chemically “bound” to humectants or solutes, such as salts, sugars, or polyols, the water is biologically unavailable for microbial growth. Water activity measures the amount of water available (unbound) for chemical and biochemical reactions, and for microbial growth to occur. Every microorganism has a limiting water activity level below which it cannot grow.

Knowledge of the water activity of pharmaceutical substances i.e., drugs and excipients is essential to obtain a product formulation with optimal chemical, physical, microbial, and shelf-life properties. Water activity influences the chemical stability, microbial stability, flow properties, compaction, hardness, and dissolution rate of dosage forms of pharmaceuticals.

### Instrument/Method for Measuring Water Activity:

Instrument: Aqualab Series 4TEV Dew Point Water Activity Meter (uses both a chilled-mirror dewpoint sensor and a capacitance sensor for measuring non-volatile and volatile substances, respectively). The instrument has the following attributes:
Water Activity Range: 0.030 to 1.000 aw
Water Activity Accuracy: +0.003 (4TE Dew Point Mode)
Water Activity Accuracy: +0.015 (4TEV Capacitance Mode)
Read Time1: ≤5 min.
Sample Temperature Range: 15 to 50° C.
Sample Temperature Accuracy: +0.2° C.
Sample Dish Capacity: 15 ml Full
Operating Environment: 5 to 50° C. 20 to 80% Humidity

Aw is performed following a verification of Aqualab's water activity calibration against known verification standards to guarantee optimal performance and accuracy. Verification standards are specially prepared unsaturated salt solutions having a specific molality and water activity value which are accurately measurable. Calibration verification is performed using two known verification standards (0.760 aw and 0.250 aw), before use.

The Aqualab water activity meter uses the chilled-mirror dewpoint technique to measure the water activity of a sample. In an instrument that uses the dewpoint technique, the sample is equilibrated with the headspace of a sealed chamber that contains a mirror and a means of detecting condensation on the mirror. At equilibrium, the relative humidity of the air in the chamber is the same as the water activity of the sample. In the AquaLab water activity meter, the mirror temperature is precisely controlled by a thermoelectric cooler. Detection of the exact point at which condensation first appears on the mirror is observed with a photo-electric cell. A beam of light is directed onto the mirror and reflected into a photo detector cell. The photo detector senses the change in reflectance when condensation occurs on the mirror. A thermocouple attached to the mirror then records the temperature at which condensation occurs. The AquaLab water activity meter then signals by beeping and displays the final water activity and temperature. In addition to the technique described above, the AquaLab water activity meter uses an internal fan that circulates the air within the sample chamber to reduce equilibrium time. Since both dewpoint and sample surface temperatures are simultaneously measured, the need for complete thermal equilibrium is eliminated, which reduces measurement times to less than five minutes.

### Examples

The compositions of the present invention described in the following examples illustrate specific embodiments of the present invention, but are not intended to be limiting thereof. Other modifications can be undertaken by the skilled artisan without departing from the spirit and scope of this invention.

### Example 1: Liquid Diphenhydramine Formulation Comprising Tapioca Syrup

The present example was prepared using the formula in Table 2, using tapioca syrup as a natural bulk sweetener.

TABLE 2
Formulation Comprising Tapioca Syrup

| | | mg/ | % W/V |
|---|---|---|---|
| Ingredient | Function | 5 ml | (g/100 ml) |
| Glycerin USP | Bulk Liquid | 1500 | 30 |
| Stevia¹ | Sweetener | 7.5 | 0.15 |
| Monoammonium Glycyrrhizinate² | Sweetener | 2 | 0.04 |
| Diphenhydramine HCl | API | 12.5 | 0.25 |
| Cherry Flavor | Flavor | 25 | 0.5 |
| Clarified Tapioca syrup³ | Bulk Sweetener | QS⁴ | QS to 100 |
| | | | ml |
| TOTAL | | | 100 mL |

¹Commercially available product under the trademark SteviaSweet ® RM95 from Icon Foods corporation
²Commercially available product under the trademark MagnaSweet ® MM100 from the MafCo Corporation
³Commercially available product under the trademark TapiSweet ® CL42 from the Sweet Additions Corporation
⁴“QS” is a term to indicate that this material was used to bring up to a final target volume

### Processing Directions

1. To a tared vessel equipped with high shear mixer, the glycerin was added to achieve suitable volume for mixing.
2. Approximately one half of the tapioca syrup was added to the vessel and mixed.
3. The stevia, monoammonium glycyrrhizinate, diphenhydramine and flavor were added and mixed until dissolved.
4. The solution was brought (“QS′d”) to final volume with tapioca syrup, and mixed under vacuum to deaerate.

### Example 2: Liquid Diphenhydramine Formulation Comprising Rice Syrup

The present example was prepared using the formula in Table 3, using brown rice syrup as a natural bulk sweetener.

TABLE 3
Formulation Comprising Brown Rice Syrup

| | | mg/ | % W/V |
|---|---|---|---|
| Ingredient | Function | 5 ml | (g/100 ml) |
| Glycerin USP | Bulk Liquid | 1500 | 30 |
| Stevia¹ | Sweetener | 1000 | 20 |
| Monoammonium Glycyrrhizinate² | Sweetener | 2 | 0.04 |
| Anhydrous Citric Acid USP | Acidulant | 2.5 | 0.05 |
| Diphenhydramine Hydrochloride | API | 12.5 | 0.25 |
| USP | | | |
| Cultured Dextrose Total (Inhibit | Flavor | 25 | 0.5 |
| Violet³) | | | |
| Brown Rice Syrup⁴ | Bulk Sweetener | 2150 | 49 |
| Cherry Flavor | Flavor | 25 | 0.5 |
| Purified Water USP | | QS⁵ | QS to 100 |
| | | | ml |
| TOTAL | | | 100 mL |

¹Commercially available product under the trademark SteviaSweet ® RM95 from Icon Foods corporation
²Commercially available product under the trademark MagnaSweet ® MM100 from the MafCo Corporation
³Commercially available from Mezzoni Foods
⁴Commercially available product under the trademark RiceSweet ® CL42 from the Sweet Additions Corporation
⁵“QS” is a term to indicate that this material was used to bring up to a final target volume

### Processing Directions

1. To a tared vessel equipped with high shear mixer, the glycerin was added to achieve suitable volume for mixing.
2. Approximately one half of the brown rice syrup was added to the vessel and mixed.
3. The stevia, monoammonium glycyrrhizinate, diphenhydramine, anhydrous citric acid and flavor were added and mixed until dissolved.
4. The solution was brought (“QS′d”) to final volume with purified water, and mixed under vacuum to deaerate.

### Example 3: Liquid Diphenhydramine Formulation Comprising Allulose Syrup

The present example was prepared using the formula in Table 4, using allulose syrup as a natural bulk sweetener.

TABLE 4
Formulation Comprising Allulose Syrup

| | | mg/ | % W/V |
|---|---|---|---|
| Ingredient | Function | 5 ml | (g/100 ml) |
| Glycerin USP | Bulk Liquid | 1500 | 30 |
| Allulose Syrup¹ | Sweetener | 10 | 0.2 |
| Monoammonium Glycyrrhizinate | Sweetener | 2 | 0.04 |
| (MagnaSweet MM100²) | | | |
| Allulose Syrup¹ | Bulk Sweetener | 2500 | 50 |
| Diphenhydramine HCl | API | 12.5 | 0.25 |
| Grape Flavor | Flavor | 25 | 0.5 |
| Allulose Syrup¹ | Bulk Sweetener | QS² | QS to 100 |
| | | | ml |
| TOTAL | | | 100.0 mL |

¹Commercially available product under trademark AllSWEET ® AG9957 from the Anderson Advanced Ingredients Corporation
²“QS” is a term to indicate that this material was used to bring up to a final target volume

### Processing Directions

1. To a tared vessel equipped with high shear mixer, the glycerin is added to achieve suitable volume for mixing.
2. Approximately one half of the allulose syrup was added to the vessel and mixed.
3. The stevia, monoammonium glycyrrhizinate, diphenhydramine, and flavor were added and mixed until dissolved.
4. The solution was brought (“QS′d”) to final volume with allulose syrup, and mixed under vacuum to deaerate.

### Example 4: Liquid Diphenhydramine Formulation Comprising Fruit-Up® Syrup

The present example was prepared using the formula in Table 5, using the combination of apple syrup and carob syrup, for example as commercially sold under the trademark Fruit-Up® by ADM Company, as a natural bulk sweetener.

TABLE 5
Formulation Comprising Fruit-Up ® Syrup

| | | Mg/ | Amount |
|---|---|---|---|
| Ingredient | Function | 5 mL | (% w/v) |
| Glycerin USP | Bulk Liquid | 1500 | 30 |
| Stevia¹ | Sweetener | 10 | 0.2 |
| Monoammonium Glycyrrhizinate² | Sweetener | 2 | 0.04 |
| Combination of Apple Syrup and | Bulk Sweetener | 2500 | 50.0 |
| Carob Syrup³ | | | |
| Diphenhydramine HCl | API | 12.5 | 0.25 |
| Grape Flavor | Flavor | 17.5 | 0.35 |
| Combination of Apple Syrup and | Bulk Sweetener | QS⁴ | QS to 100 |
| Carob Syrup³ | | | ml |

¹Commercially available product under the trademark AudianaSweet ® Optimized AG3240from the Anderson Advanced Ingredients Corporation
²Commercially available product under the trademark MagnaSweet ® MM100 from the MafCo Corporation
³Commercially available product under trademark Fruit-Up ® Premium from the ADM Company
⁴“QS” is a term to indicate that this material was used to bring up to a final target volume

### Processing Directions

1. To a tared vessel equipped with high shear mixer, the Fruit-Up® is added to achieve suitable volume for mixing.
2. Approximately one half of the fruit syrup was added to the vessel and mixed.
3. The stevia, monoammonium glycyrrhizinate, diphenhydramine, and flavor were added and mixed until dissolved.
4. The solution was brought (“QS′d”) to final volume with Fruit-Up® syrup, and mixed under vacuum to deaerate.

### Example 5: Liquid Diphenhydramine Formulation Comprising Fruit-Up® Syrup, with a ratio of Fruit-Up® Syrup: Glycerin 3.30:1

The present example was prepared using the formula in Table 6, using the combination of apple syrup and carob syrup, for example as commercially sold under the trademark Fruit-Up® by ADM Company, as a natural bulk sweetener.

TABLE 6
Formulation Comprising Fruit-Up ® Syrup

| | | Mg/ | Amount | Amount |
|---|---|---|---|---|
| Ingredient | Function | 5 mL | (% w/v)³ | (% w/w)⁴ |
| Glycerin USP | Bulk Liquid | 1500.03 | 30.00 | 23.145 |
| Combination of Apple | Bulk | 4945.78 | 98.92 | 76.312 |
| Syrup and Carob Syrup³ | Sweetener | | | |
| Diphenhydramine HCl | API | 12.51 | 0.25 | 0.193 |
| Sodium Citrate Dihydrate | pH Adjusting | 6.48 | 0.13 | 0.100 |
| USP | agent | | | |
| Natural Flavor | Flavor | 16.20 | 0.32 | 0.250 |
| Combination of Apple | Bulk | QS² | QS to 100 | |
| Syrup and Carob Syrup³ | Sweetener | | ml | |
| TOTAL | | | 129.620 | 100.00 |

¹Commercially available product under trademark Fruit-Up ® Premium from the ADM Company
²“QS” is a term to indicate that this material was used to bring up to a final target volume
³% w/v = % weight/volume
⁴% w/w = % weight/weight

### Processing Directions

1. To a tared vessel equipped with high shear mixer, the Fruit-Up® is added to achieve suitable volume for mixing.
2. Approximately one half of the Fruit-Up® syrup was added to the vessel and mixed.
3. The sodium citrate dihydrate, diphenhydramine and flavor were added and mixed until dissolved.
4. The solution was brought (“QS′d”) to final volume with Fruit-Up® syrup, and mixed under vacuum to deaerate.

### Example 6: Liquid Cetirizine Formulation Comprising Tapioca Syrup

The present example is prepared using the formula in Table 7, using tapioca syrup as a natural bulk sweetener.

TABLE 7
Cetirizine Formulation Comprising Tapioca Syrup

| | | mg/ | % W/V |
|---|---|---|---|
| Ingredient | Function | 5 ml | (g/100 ml) |
| Glycerin USP | Bulk Liquid | 1500 | 30 |
| Stevia¹ | Sweetener | 7.5 | 0.15 |
| Monoammonium Glycyrrhizinate² | Sweetener | 2 | 0.04 |
| Cetirizine HCl | API | 5.0 | 0.5 |
| Cherry Flavor | Flavor | 25 | 2.5 |
| Clarified Tapioca syrup³ | Bulk Sweetener | QS⁴ | QS to 100 |
| | | | ml |
| TOTAL | | | 100 mL |

¹Commercially available product under trademark SteviaSweet ® RM95 from the Anderson Advanced Ingredients Corporation
²Commercially available product under trademark MagnaSweet ® MM100 from the MafCo Corporation
³Commercially available product under trademark TapiSweet ® CL42 from the Sweet Additions Corporation
⁴“QS” is a term to indicate that this material was used to bring up to a final target volume

### Processing Directions

1. To a tared vessel equipped with high shear mixer, the glycerin is added to achieve suitable volume for mixing.
2. Approximately one half of the tapioca syrup was added to the vessel and mixed.
3. The stevia, monoammonium glycyrrhizinate, cetirizine HCl, and flavor were added and mixed until dissolved.
4. The solution was brought (“QS′d”) to final volume with tapioca syrup, and mixed under vacuum to deaerate.

### Example 7: Liquid Loperamide Formulation Comprising Tapioca Syrup

The present example was prepared using the formula in Table 8, using tapioca syrup as a natural bulk sweetener.

TABLE 8
Loperamide Formulation Comprising Tapioca Syrup

| | | mg/ | % W/V |
|---|---|---|---|
| Ingredient | Function | 5 ml | (g/100 ml) |
| Glycerin USP | Bulk Liquid | 1500 | 30 |
| Stevia¹ | Sweetener | 7.5 | 0.15 |
| Monoammonium Glycyrrhizinate² | Sweetener | 2 | 0.04 |
| Loperamide HCl | API | 5.0 | 0.5 |
| Cherry Flavor | Flavor | 25 | 2.5 |
| Clarified Tapioca syrup³ | Bulk Sweetener | QS⁴ | QS to 100 |
| | | | ml |
| TOTAL | | | 100 mL |

¹Commercially available product under trademark SteviaSweet ® RM95 from the Anderson Advanced Ingredients Corporation
²Commercially available product under trademark MagnaSweet ® MM100 from the MafCo Corporation
³Commercially available product under trademark TapiSweet ® CL42 from the Sweet Additions Corporation
⁴“QS” is a term to indicate that this material was used to bring up to a final target volume

### Processing Directions

1. To a tared vessel equipped with high shear mixer, the glycerin is added to achieve suitable volume for mixing.
2. Approximately one half of the tapioca syrup was added to the vessel and mixed.
3. The stevia, monoammonium glycyrrhizinate, loperamide HCl, and flavor were added and mixed until dissolved.
4. The solution was brought (“QS′d”) to final volume with tapioca syrup, and mixed under vacuum to deaerate.

### Example 8: Liquid Loratadine Formulation Comprising Fruit-Up® Syrup Natural Bulk Sweetener

The present example was prepared using the formula in Table 9, using the combination of apple syrup and carob syrup, for example as commercially sold under the trademark Fruit-Up® by ADM Company, as a natural bulk sweetener.

TABLE 9
Formulation Comprising Fruit-Up ® Syrup

| | | Mg/ | Amount |
|---|---|---|---|
| Ingredient | Function | 5 mL | (% w/v) |
| Glycerin USP | Bulk Liquid | 1500 | 30 |
| Stevia¹ | Sweetener | 10 | 0.2 |
| Monoammonium Glycyrrhizinate² | Sweetener | 2 | 0.04 |
| Combination of Apple Syrup and | Bulk Sweetener | 2500 | 50.0 |
| Carob Syrup³ | | | |
| Loratadine | API | 5.00 | 0.1 |
| Grape Flavor | Flavor | 17.5 | 0.35 |
| Combination of Apple Syrup and | Bulk Sweetener | QS⁴ | QS to 100 |
| Carob Syrup³ | | | ml |

¹Commercially available product under trademark AudianaSweet ® Optimized AG3240 from the Anderson Advanced Ingredients Corporation
²Commercially available product under trademark MagnaSweet ® MM100² from the MafCo Corporation
³Commercially available product under trademark Fruit-Up ® Premium from the ADM Company
⁴“QS”is a term to indicate that this material was used to bring up to a final target volume

### Processing Directions

1. To a tared vessel equipped with high shear mixer, the glycerin is added to achieve suitable volume for mixing.
2. Approximately one half of the Frit-Up syrup was added to the vessel and mixed.
3. The stevia, monoammonium glycyrrhizinate, loratadine, and flavor were added and mixed until dissolved.

### Example 9: Rheology (Viscosity) Procedure

The viscosity data presented in the table shows a snapshot of measured viscosity at various shear rates. The viscosity measurement was carried out using a Brookfield DV2T LV type viscometer coupled with a small sample adapter kit and spindle #31. A measurement temperature of 25° C. was maintained with a temperature control water bath for all the samples. For each measurement 9 mL of sample was loaded without air bubbles. Spindle rotation speed was set at 20 RPM and the readings were recorded in units of cps after 5 rotations of the spindle. The viscosity of Example 5 was measured to be about 328.5 centipoise.

### Shear Sweep

All tests were performed using a TA Instruments AR2000 rheometer with a 60 mm parallel plate geometry with gap setting of 0.5 mm. Measurement temperature of 25° C. was maintained with a Peltier plate for all the samples. The rheometer was mounted on a vibration-isolated platform and leveled. Samples were loaded without air bubbles and excess sample was “trimmed” from the edges when the gap was 10% larger than the desired gap. Shear sweep experiments were run to with shear rates ranging from 0.001 to 100 l/s.

pH

The pH of Example 5 was measured to be about 5.88.

### Example 10: Assay and Stability

The formula from Example 5 was tested for assay of diphenhydramine and tested on stability at the following timepoints: Initial (Time 0) and 1 month and stored at various conditions as shown in Table 10. The amount of total diphenhydramine and degradation products were analyzed using a reference standard prepared at 100% of the label claimed dose. Samples and standard were diluted and mixed in a diluent consisting of 50 mM ammonium formate plus 0.1% formic acid in water: methanol at a ratio of 95:5

Method: The diphenhydramine assays and degradants were analyzed using ultra high pressure liquid chromatography (UPLC) with the following parameters:
Column: Waters Acquity UPLC HSS T3 2.1×150, 1.8 μm

Mobile Phase: Gradient method of a first mobile phase of 30 mM Potassium Hexafluorophosphate plus 0.05% Phosphoric Acid in Water and a second mobile phase of Acetonitrile: Water at a ratio of 80:20 plus 0.05% Phosphoric Acid over a run time of 14 minutes. The method was run with a flow rate of 0.45 mL/min, an injection volume of 2.0 μL, a UV Detector at 220 nm and a column temperature of 40° C. The suggested run time was 14 minutes.

TABLE 10
Assay and Stability of Example 5

| Time Point (Months, | Diphenhydramine | Total Degradation | Density |
|---|---|---|---|
| Storage Condition) | Assay (%) | products (%) | (g/mL) |
| 0 (Initial) | 101.1 | ND¹ | 1.3235 |
| 1 month, 25° C. | 102.0 | ND | 1.3275 |
| 1 month, 40° C. | 101.5 | ND | 1.3273 |
| 1 month, 5° C. | 101.6 | ND | 1.3273 |

¹ND = None Detected

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

1. A liquid composition, wherein the liquid composition comprises at least one therapeutic ingredient and at least one natural bulk sweetener wherein the fructose content of the total amount of natural bulk sweetener in the composition is less than 75 percent.

2. The liquid composition of claim 1, wherein the natural bulk sweetener is selected from glycerin, allulose syrup, rice syrup, brown rice syrup, tapioca syrup, apple syrup, carob syrup, and combinations thereof.

3. The liquid composition of claim 1, wherein the liquid composition has a total fructose level of less than 0.7 g/mL.

4. The liquid composition of claim 1, wherein the therapeutic ingredient is an pharmaceutical active ingredient.

5. The liquid composition of claim 1, wherein the therapeutic ingredient is a homeopathic active ingredient.

6. The liquid composition of claim 1, wherein the therapeutic ingredient is a nutritional supplement.

7. The liquid composition of claim 1, wherein the liquid composition is in the form of a suspension.

8. The liquid composition of claim 1, wherein the liquid composition is the form of a solution.

9. The liquid composition of claim 4, wherein the pharmaceutical active ingredient is selected from acetaminophen, diphenhydramine, dextromethorphan, guaifenesin, ibuprofen, cetirizine, loperamide and phenylephrine.

10. The liquid composition of claim 1, wherein the therapeutic ingredient is an antihistamine.

11. The liquid composition of claim 1, further comprising a sensate.

12. The liquid composition of claim 1, further comprising a second natural bulk sweetener.

13. The liquid composition of claim 12, wherein the natural bulk sweetener is glycerin and the second natural bulk sweetener is tapioca syrup.

14. The liquid composition of claim 12, wherein the natural bulk sweetener is glycerin and the second natural bulk sweetener is brown rice syrup.

15. The liquid composition of claim 12, wherein the natural bulk sweetener is glycerin and the second natural bulk sweetener is allulose syrup.

16. The liquid composition of claim 12, wherein the natural bulk sweetener is glycerin and the second natural bulk sweetener is a combination of apple syrup and carob syrup.

17. The liquid composition of claim 16, wherein the ratio of the combination of apple syrup and carob syrup to glycerin is about 3.30:1.

18. The liquid composition of claim 1, wherein the liquid composition is substantially free of agave syrup.

19. The liquid composition of claim 1, wherein the liquid composition is substantially free of any ingredient having a fructose content greater than 15 percent.

20. A method of alleviating a symptom selected from pain, fever, cough, nasal congestion, diarrhea, allergic rhinitis and sore throat in a subject comprising administering the composition of claim 1.
